(19) United States
(12) Patent Application Publication
(10) Pub. No.: US 2007/0265494 A1
(43) Pub. Date: Nov. 15, 2007

(54) Flexible and retractable endoscope elevator

(75) Inventors: Gary J. Leanna, Holden, MA (US); Kurt Geitz, Sudbury, MA (US)

Correspondence Address: FINNEGAN, HENDERSON, FARABOW, GARRETT & DUNNER;LLP, 901 NEW YORK AVENUE, NW, WASHINGTON, DC, 20001-4413, US

(73) Assignee: Boston Scientific Scimed Inc.

(21) Appl. No.: 11/430,831

(22) Filed: May 10, 2006

(51) Int. Cl. A61B 1/00

(52) U.S. Cl. 600/107; 600/104

(57) ABSTRACT

Embodiments of the invention include a medical device for accessing a patient's body portion and used for diagnosis and treatment of medical conditions. Embodiments of the invention may include a particular endoscopic positioning mechanism for placing an endoscope and an additional treatment device within desired body portions in order to assist in diagnosis and treatment of anatomical diseases and disorders. In particular, a medical device according to an embodiment of the invention includes an elongated elevator configured to receive and direct a treatment instrument for placement at a treatment location.

### FIELD OF THE INVENTION

The invention relates to an endoscope system for accessing a patient's body portion and used for diagnosis and treatment of medical conditions. For example, embodiments of the invention may include a particular endoscopic positioning mechanism for placing an endoscope and an additional treatment device within desired body portions in order to assist in diagnosis and treatment of anatomical diseases and disorders.

### BACKGROUND OF THE INVENTION

Endoscopes for medical use have been adopted for various diagnostic and medical treatment procedures. Endoscopes have been used for the diagnosis and treatment of a wide range of diseases and disorders that often require a physician to access the tortuous and relatively small cross-sectional areas of a patient's internal anatomical body lumens. A patient's pancreaticobiliary system (including the anatomical regions of the gall bladder, pancreas, and the biliary tree), for example, is accessed for diagnosis, and/or treatment of disorders of certain portions of the digestive system.

During treatment of the digestive system, endoscopes are often used to access and visualize a patient's pancreaticobiliary system. Once the endoscope is positioned in the desired body portion, a treatment instrument can be advanced through the working channel of the endoscope to the desired body portion. The endoscope and treatment instrument may then be manipulated as desired for visualization and treatment respectively.

Endoscopic retrograde cholangiopancreatography (ERCP) is one example of a medical procedure that uses an endoscope. ERCP enables the physician to diagnose problems in the liver, gallbladder, bile ducts, and pancreas. The liver is a large organ that, among other things, makes a liquid called bile that helps with digestion. The gallbladder is a small, pear-shaped organ that stores bile until it is needed for digestion. The bile ducts are tubes that carry bile from the liver to the gallbladder and small intestine. These ducts are sometimes called the biliary tree. The pancreas is a large gland that produces chemicals that help with digestion and hormones such as insulin.

The biliary system delivers bile produced by the liver to the duodenum where the bile assists other gastric fluids in digesting food. The biliary system includes the liver, as well as a plurality of bodily channels and organs that are disposed between the liver and the duodenum. Within the liver lobules, there are many fine “bile canals” that receive secretions from the hepatic cells. The canals of neighboring lobules unite to form larger ducts, and these converge to become the “hepatic ducts.” They merge, in turn, to form the “common hepatic duct.” The “common bile duct” is formed by the union of the common hepatic and the cystic ducts. It leads to the duodenum, where its exit is guarded by a sphincter muscle. This sphincter normally remains contracted until the bile is needed, so that bile collects in the common bile duct and backs up to the cystic duct. When this happens, the bile flows into the gallbladder and is stored there.

ERCP is used primarily to diagnose and treat conditions of the bile ducts, including gallstones, inflammatory strictures, leaks (from trauma and surgery), and cancer. ERCP combines the use of x-rays and an endoscope. Through the endoscope, the physician can see the inside of the stomach and duodenum, and inject dyes into the ducts in the biliary tree and pancreas so they can be seen on x-rays.

An ERCP is performed primarily to identify and/or correct a problem in the bile ducts or pancreas. For example, if a gallstone is found during the exam, it can often be removed by means of a treatment instrument, eliminating the need for major surgery. If a blockage in the bile duct causes yellow jaundice or pain, it can be relieved through the use of a treatment instrument inserted through the endoscope.

Since endoscopes are often used to access the tortuous and relatively small cross-sectional areas of a patient's internal anatomical body lumens, repeated manipulation and positioning of an endoscope during a medical procedure can cause problematic side-effects. For example, repeated manipulation and positioning of the endoscope can cause unnecessary trauma to a patient's internal tissues. Improper placement and repeated attempts to access a desired treatment region can exacerbate tissue trauma as well as unnecessarily prolong the medical procedure. Accordingly, there is a need for more precise endoscope manipulation as well as manipulating an underlying treatment instrument through an access channel of an endoscope.

Thus, it is desirable to have an endoscope assembly that can more precisely access the tortuous and relatively small cross-sectional areas of certain anatomical body lumens, and more precisely manipulate a treatment device provided within an access channel of an endoscope.

### SUMMARY OF THE INVENTION

Embodiments of the present invention are directed to an improved endoscope system and a positioning device for manipulating a treatment device that obviates one or more of the limitations and disadvantages of prior medical devices. In one embodiment, a medical device comprises an elongated flexible tube including a distal end and a proximal end defining a longitudinal axis and a channel extending from the proximal end to an aperture proximate to the distal end. An elongated elevator is positioned within the channel and movable relative to the channel. The elevator has a first shape when the elevator is within the channel of the tube and a second shape when the elevator extends from the aperture beyond the channel. The elevator is configured to receive and direct a treatment instrument for placement at a treatment location.

In various embodiments, the device may include one or more of the following additional features: wherein the elevator is formed of a shape memory material; wherein the elevator is configured to retain the second shape without the application of a force to the elevator; wherein the elevator is configured to retain the first shape when a force is applied to the elevator; wherein the force is applied by the tube; wherein the first shape of the elevator corresponds to an internal shape of the channel in the tube and the second shape of the elevator comprises a curved shape; wherein the aperture is a side facing aperture opening laterally along the flexible tube and the curved shape comprises a curve which deflects proximally relative to the flexible tube; wherein the aperture is a side facing aperture opening laterally along the flexible tube and the curved shape comprises a curve which deflects distally relative to the flexible tube; wherein the second shape of the elevator comprises a distal linear portion and an intermediate curved portion; wherein the intermediate curved portion comprises a curve extending at least 90 degrees and exhibiting a substantially constant radius of curvature; wherein the elevator has a V-shaped cross-section; wherein the elevator has a U-shaped cross-section; wherein the elevator has a recess channel configured to receive an external surface of a treatment instrument therein; wherein the elevator is configured for sideways deflection when extended beyond the channel of the tube through actuation of a pull wire connected to a distal portion of the elevator and extending proximally within the medical device; wherein two pull wires are connected to a distal portion of the elevator and extend proximally within laterally offset lumens within the tube; wherein the medical device is an endoscope that includes visualization and illumination components therein; wherein the medical device is an endoscope that includes an additional positioning mechanism for achieving controlled deflection of the elongated flexible tube; a handle at the proximal end of the flexible tube, the handle including a positioning mechanism connected to the elevator and for extending and retracting the elevator within the channel of the flexible tube; wherein the positioning mechanism comprises a slide block connected to a proximal portion of the elevator, the slide block extending within a slide channel formed along an exterior surface of the handle; wherein the slide channel includes boundaries limiting the distance the slide block can extend and retract the elevator; wherein the positioning mechanism includes a locking mechanism for fixing the position of the elevator relative to the channel of the flexible tube; wherein the elevator exhibits a third shape when the elevator extends from the distal end of the tube beyond the channel and a restraining force is removed from the elevator; wherein the first shape of the elevator corresponds to an internal shape of the channel in the tube and the second shape of the elevator comprises a curved shape and the third shape comprises a shape having a greater curve than the second shape; wherein the elevator comprises a hollow tube configured for receiving a treatment instrument therein; wherein the elevator comprises a hollow tube slidably received within a sleeve; wherein the sleeve imparts a predetermined rigidity to the hollow tube when surrounding the hollow tube, thereby restraining the hollow tube to exhibit the second elevator shape when the elevator extends from the distal end of the elongated flexible tube beyond the channel; wherein upon extension of the elevator beyond the elongated flexible tube and upon retraction of the sleeve relative to the hollow tube, the elevator exhibits the third shape; and wherein the first shape of the elevator corresponds to an internal shape of the channel in the tube and the second shape of the elevator comprises a curved shape and the third shape comprises a shape having a lateral bend.

Another embodiment is directed to a method of positioning a treatment instrument in a body. The method comprises providing a medical device including an elongated flexible tube including a distal end and a proximal end defining a longitudinal axis and a channel extending from the proximal end to an aperture at the distal end. An elongated elevator is positioned within the channel and movable relative to the channel. The elevator has a first shape when the elevator is within the channel of the tube and a second shape when the elevator extends from the distal end of the tube beyond the channel. The elevator is configured to receive and direct a treatment instrument for placement at a treatment location. The method also includes inserting the medical device into an anatomical lumen of the body, extending the elevator beyond the channel of the tube such that the elevator achieves the second shape, and inserting a treatment instrument along the elevator such that the treatment instrument is positioned at a treatment site.

In various embodiments, the method may include one or more of the following additional features: retracting the treatment instrument into the medical device, retracting the elevator into the channel of the tube, repositioning the medical device within the anatomical lumen, and redeploying the elevator and treatment instrument; wherein the medical device includes a handle having a positioning mechanism for extending, retracting, and locking the position of the elevator within the channel of the flexible tube; and further comprising locking the position of the elevator within the channel of the flexible tube; wherein the treatment instrument is positioned within a bile duct during an ERCP procedure; wherein the medical device is an endoscope that includes visualization and illumination components therein; wherein the medical device is an endoscope that includes an additional positioning mechanism for achieving controlled deflection of the elongated flexible tube; wherein the elevator is formed of a shape memory material; wherein the elevator is configured to retain the second shape without the application of a force to the elevator; wherein the elevator is configured to retain the first shape when a force is applied to the elevator; wherein the force is applied by the tube; wherein the first shape of the elevator corresponds to an internal shape of the channel in the tube and the second shape of the elevator comprises a curved shape; wherein the elevator has a V-shaped cross-section; wherein the elevator has a U-shaped cross-section; wherein the elevator is configured for sideways deflection when extended beyond the channel of the tube through actuation of a pull wire connected to a distal portion of the elevator and extending proximally within the medical device; wherein two pull wires are connected to a distal portion of the elevator and extend proximally within laterally offset lumens within the tube; wherein the elevator exhibits a third shape when the elevator extends from the distal end of the tube beyond the channel and a restraining force is removed from the elevator; wherein the elevator comprises a hollow tube slidably received within a sleeve; wherein the sleeve imparts a predetermined rigidity to the hollow tube when surrounding the hollow tube, thereby restraining the hollow tube to exhibit the second elevator shape when the elevator extends from the distal end of the elongated flexible tube beyond the channel; further comprising extending the elevator beyond the elongated flexible tube and retracting the sleeve relative to the hollow tube such that the elevator exhibits the third shape; and wherein the first shape of the elevator corresponds to an internal shape of the channel in the tube and the second shape of the elevator comprises a curved shape and the third shape comprises a shape having a lateral bend.

Additional objects and advantages of the invention will be set forth in part in the description which follows, and in part will be obvious from the description, or may be learned by practice of the invention. The objects and advantages of the invention will be realized and attained by means of the elements and combinations particularly pointed out in the appended claims.

It is to be understood that both the foregoing general description and the following detailed description are exemplary and explanatory only and are not restrictive of the invention, as claimed.

The accompanying drawings, which are incorporated in and constitute a part of this specification, illustrate several embodiments of the invention and together with the description, serve to explain the principles of the invention.

### BRIEF DESCRIPTION OF THE DRAWINGS

FIG.1 is a perspective view of a prior art endoscope system.

FIG. 2 is a cross-sectional view illustrating the structure of a known elevator device.

FIG. 3 is a cross-sectional view of a distal portion of an endoscope according to an embodiment of the present invention.

FIG. 4 illustrates a proximal portion of an endoscope according to an embodiment of the present invention.

FIG. 5 is a perspective view of an exemplary elevator according to an embodiment of the present invention.

FIG. 6 is a cross-sectional view of a distal portion of an endoscope illustrating a partially deployed elevator according to an embodiment of the present invention.

FIG. 7 is a cross-sectional view of a distal portion of an endoscope illustrating a partially deployed elevator according to an embodiment of the present invention.

FIG. 8A is a partial cross-sectional view of a distal portion of an endoscope illustrating a partially deployed elevator according to an embodiment of the present invention.

FIG. 8B is a partial cross-sectional view of a distal portion of an endoscope illustrating a more fully deployed elevator according to an embodiment of the present invention.

FIG. 9A depicts a top view of an additional elevator arrangement according to an embodiment of the present invention.

FIG. 9B depicts a top view of the elevator arrangement of FIG. 9A in a more fully deployed configuration according to an embodiment of the present invention.

FIGS. 10 and 11 are cross-sectional views of a distal portion of an endoscope according to another embodiment of the present invention.

FIGS. 12A-12C are cross-sectional views of various configurations of elevators according to embodiments of the present invention.

FIG. 13 illustrates the positioning of an endoscope and treatment device within a patient's body portion according to an embodiment of the invention.

### DESCRIPTION OF THE EMBODIMENTS

Reference will now be made in detail to the exemplary embodiments of the invention, examples of which are illustrated in the accompanying drawings. Wherever possible, the same reference numbers will be used throughout the drawings to refer to the same or like parts. The drawing figures of this application are intended to provide a general understanding of the working elements of the underlying system. Accordingly, unless explicitly stated, the figures do not represent a literal depiction of proportional dimensions or the precise locations for the illustrated inter-related components.

According to exemplary embodiments, the invention relates to a medical device for positioning a treatment device and/or viewing a patient's internal body portion. In embodiments that use a treatment device in an endoscopic medical procedure, the treatment device can be advanced through a working channel of an endoscope, including an endoscope specifically designed and/or sized for use with the treatment device, and into a tissue tract. For purposes of this disclosure, “treatment device” or “treatment instrument” includes, for example, any working medical device advanced through a working channel of an endoscope and for use during an endoscopic procedure. Exemplary treatment instruments include, but are not limited to, guide wires, cutting or grasping forceps, biopsy devices, snare loops, injection needles, cutting blades, scissors, retractable baskets, retrieval devices, ablation and/or electrophysiology catheters, stent placement devices, surgical stapling devices, and balloon catheters.

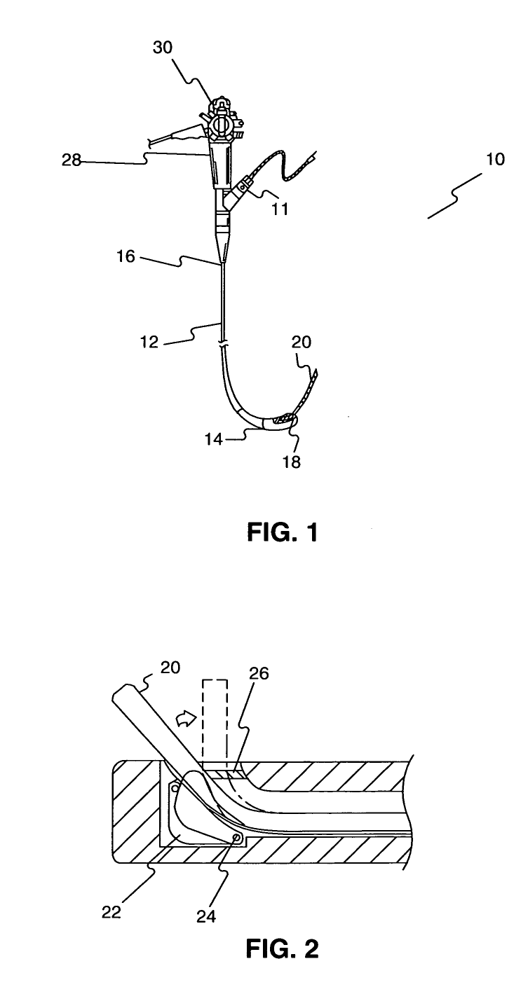

FIG. 1 illustrates a known endoscope system. For purposes of this disclosure, “distal” refers to the end further from the device operator during use and “proximal” refers to the end closer to the device operator during use. FIG. 1 depicts an endoscope 10 including a flexible outer tube 12 extending between a distal end 14 and a proximal end 16 of the device. Endoscope 10 includes a treatment device insertion port 11 for receiving a treatment device 20 into a working channel of the endoscope 10. The distal end 14 of the endoscope system 10 includes a side facing operation window 18 that can include visualization and lighting components for viewing during a treatment procedure. In addition, a working channel (not shown) extends within the endoscope 10 and terminates at the operation window 18, thereby allowing the treatment instrument 20 to be extended from the distal end of the endoscope 10. The extension of the treatment instrument 20 at a desired treatment site can be then viewed through the visualization components, which transmit images to the proximal end of the endoscope 10, as known in the art. While FIG. 1 illustrates a side facing operation window 18, both front/forward facing and oblique/intermediate angled windows are known.

FIG. 2 illustrates a cross-sectional view of a distal portion of a known endoscope system including a deflecting lever/elevator device for deflecting a treatment instrument as the instrument is extended beyond a working channel of an endoscope. As seen in FIG. 2, a deflecting lever 22 is rotated clockwise about a pin 24 by means of a pull wire 26 connected to an upper portion of the deflecting lever 22. Upon actuation of the pull wire 26 through proximal movement thereof, the deflecting lever 22 deflects the treatment device 20 in order to alter the angle at which the treatment device 20 exits the endoscope's working channel, resulting in the position of device 20 shown by the dashed lines in FIG. 2. By means of pull wire 26, the endoscope operator can control the placement of the treatment instrument 20 as it is positioned during a medical procedure.

As seen in FIG. 1, a handle 28 at the proximal end 16 of the device can include various positioning controls 30 to effectuate bending and rotation of the flexible outer tube 12 for positioning of the device during a medical procedure. In addition, the handle can include a distinct positioning control for actuation of the deflection lever pull wire 26. During a medical procedure such as, for example, an ERCP procedure, the treatment instrument 20 must be precisely inserted into a particular duct in the biliary tree. While the use of a deflection lever 22 is capable of altering the angle at which the treatment device exits the endoscope, precise positioning often requires repeated manipulation of the distal end of the endoscope including the operation window in order to achieve proper placement of the treatment device 20. As noted above, this repeated manipulation of the underlying endoscope 10 can lead to tissue trauma and unnecessarily prolong the entire medical procedure.

As seen in the embodiment of FIG. 2, the deflection lever 22 is displaceable about a single axis (i.e. the axis coincident with the pin 24). Rotation of lever 22 is achieved by generating a moment about the axis of pin 24 through proximal actuation of pull wire 26. The size of the moment arm for rotating the lever 22 is limited by the size of the underlying endoscope body. For example, the magnitude of the moment arm generated through actuation of the pull wire 26 is limited by the available distance the attachment point of pull wire 26 can be spaced from the pin 24. The available spacing distance, in turn, is limited by the relatively small outer diameters necessary for endoscopes capable of accessing an internal body portion. In the resulting deflection lever arrangements, accurate deflection of a lever 22 requires the generation of a relatively large pull wire actuation force. Transmitting such inordinate pull wire actuation forces along the longitudinal axis of an endoscope can interfere with accurate positioning of the underlying endoscope system during a treatment procedure. In some instances, tension can be improperly transferred to the outer flexible tube of the endoscope, which interferes with proper positioning of the endoscope.

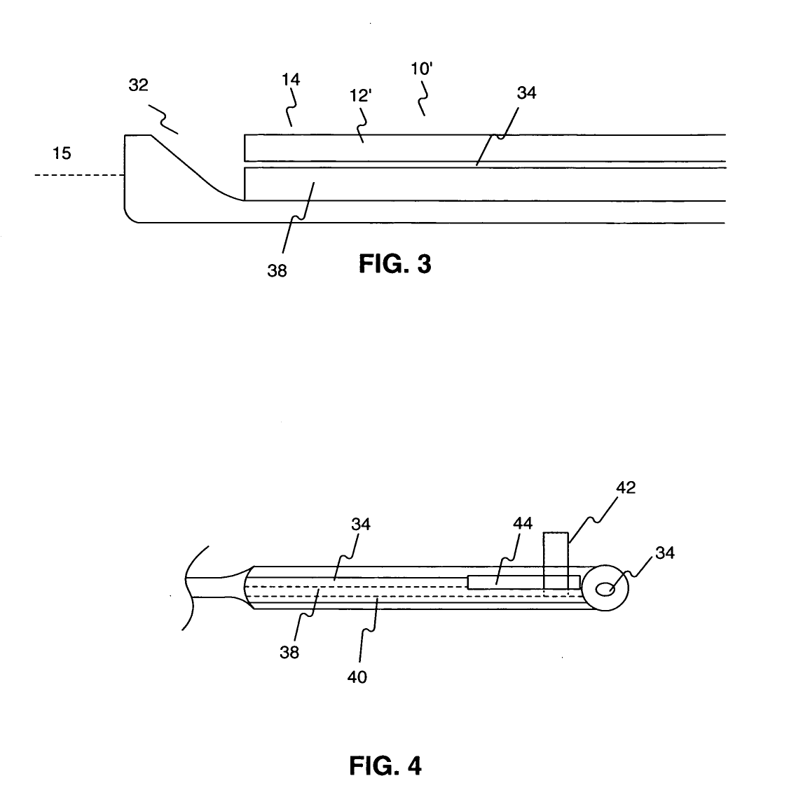

FIG. 3 depicts a cross-sectional view of a distal end 14 of an improved endoscope 10′. The distal portion of endoscope 10′ includes an exterior flexible outer tube 12′, a side facing operation window aperture 32, and a working channel 34 forming a lumen within the endoscope 10′ and extending from the proximal end of the endoscope 10′ and terminating at the operation window aperture 32. The flexible outer tube 12′ extends along a longitudinal axis 15. The working channel 34 is configured to receive a treatment instrument therein. As seen in FIG. 3, the distal end of channel 34 is curved and ramped laterally leading to aperture 32. Accordingly, during a treatment procedure, a treatment instrument can be advanced though the channel 34 until a distal end of the treatment instrument is deployed at a treatment site beyond the aperture 32. While FIG. 3 illustrates a side facing operation aperture 32, both forward facing and oblique angled embodiments are contemplated.

Channel 34 houses a retractable elongated endoscope elevator 38 therein. Elevator 38 may extend or retract within and relative to channel 34. The elevator 38 is configured to provide a guide path for a treatment instrument in order to alter the path through which the treatment instrument extends outside the endoscope's working channel 34 and aperture 32. As will be described in more detail below, the elongated elevator 38 can be configured so as to form a groove or guide channel along a surface thereof. The guide channel of elevator 38 serves to receive and guide a treatment instrument therein and, in particular, directs the placement of a treatment instrument beyond the aperture 32 during a medical procedure.

The retractable elevator 38 can be made at least partially of a shape-memory material. Shape-memory material is a material that can be formed into a particular shape, retain that shape during resting conditions (e.g., when the shaped material is in free space or when external forces applied to the shaped material are insufficient to substantially deform the shape), be deformed into a second shape when subjected to a sufficiently strong external force, and revert substantially back to the initial shape when external forces are no longer applied. Examples of shape memory materials include synthetic plastics, stainless steel, and superelastic, metallic alloys of nickel/titanium (commonly referred to as nitinol), copper, cobalt, vanadium, chromium, iron, or the like.

The elevator 38, for example, can be formed of an elongated shape memory material sized for slidable movement within the working channel 34. The elevator 38 can extend within the working channel 34 and proximally run the length of the endoscope body where it connects to a positioning mechanism for control by an operator. With reference to FIG. 4, a handle 40 at the proximal end of the endoscope 10′ can include a slide block 42 connected to a proximal portion of the elongated elevator 38. As seen in FIG. 4, the slide block 42 extends through a slide channel 44 formed along the exterior of the handle 40. Distal and proximal movement of slide block 42 relative to the exterior surface of the handle 40, effectuates distal and proximal movement of the elongated elevator 38 within the working channel 34. The length of slide channel 44 is selected to regulate the distance the elongated elevator 38 is displaced within the working channel 34 and the potential distance the elongated elevator 38 can be deployed beyond the aperture 32. In addition, the slide block 42 can incorporate a locking mechanism for releasably fixing the position of elevator 38 relative to the working channel 34. Exemplary locking mechanisms include, but are not limited to, a constricting rubber grommet mechanism, and an extendable pin and receiving aperture arrangement configured for mating engagement along a proximal end of the device. Alternatively, the operator can simply maintain the position of elevator 38 relative to the working channel 34 by hand. While a slide block and slide channel arrangement is illustrated, alternative handle positioning configurations are also contemplated. For example, the elongated elevator 38 can be extended and retracted by a worm gear arrangement, a rack and pinion arrangement, or any alternative mechanism for effectuating longitudinal displacement of an elongated component.

As noted above, the elongated elevator 38 can comprise a shape-memory material formed into a “trained” shape that is retained during resting conditions (e.g., when the shaped material is in free space or when external forces applied to the shaped material are insufficient to substantially deform the shape). Elevator 38 can be deformed into a second shape when subjected to a sufficiently strong external force and revert substantially back to the initial, trained shape when external forces are no longer applied. Referring to FIG. 3, for example, the elongated elevator 38 is illustrated in a fully retracted position within working channel 34. In this position, the elevator 38 is deformed by the internal surface of working channel 34 into a second shape that closely conforms to the shape of the working channel 34.

Upon extension beyond the working channel 34 of the endoscope 10′, the deployed portion of the elevator 38 reverts to an unrestrained trained shape. An operator can then lock the longitudinal position of the elevator 38 in the desired deployed position. The operator can then track a treatment instrument through a channel formed by the elevator 38 in order to precisely direct a treatment instrument along the path formed by the deployed shape of the exposed portion of elevator 38. Accordingly, the placement of a treatment instrument can be accomplished without repeated movement and positioning of the underlying endoscope 10′.

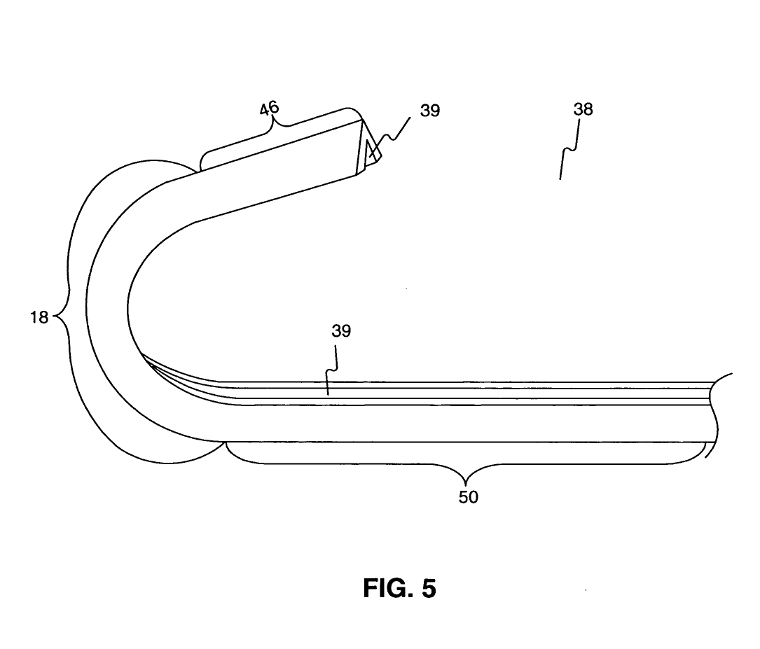

As a greater portion of elevator 38 is extended beyond the constraints of the working channel 34, the trained shape of the exposed portion may change. One possible trained shape for elevator device 38 is illustrated in FIG. 5. The particular elevator 38 of FIG. 5 is depicted in its unrestrained, trained shape. In the illustrated embodiment, the elevator 38 comprises a shape memory material exhibiting a V-shaped cross-section. The V-shape forms an internal guide channel, or conduit 39 therein. As noted above, the internal guide channel 39 of elevator 38 is configured to receive a treatment instrument therein to precisely direct it along the path formed by the deployed shape of the exposed portion of elevator 38. In one embodiment, elevator 38 is comprised of three distinct shaped segments. The distal-most segment 46 exhibits a relatively straight portion of elevator 38. The intermediate segment 48 exhibits a curved shape. The curve of intermediate segment 48 may exhibit a constant radius of curvature throughout the shape of the curve or may exhibit a variable radius of curvature. The remaining proximal segment 50 of elevator 38 exhibits a relatively linear trained shape. Since the elevator 38 of FIG. 5 is formed of a shape-memory material, it is capable of being deformed within a working channel 34 (see FIG. 3) of an endoscope 10′ where it conforms to the internal shape of the channel.

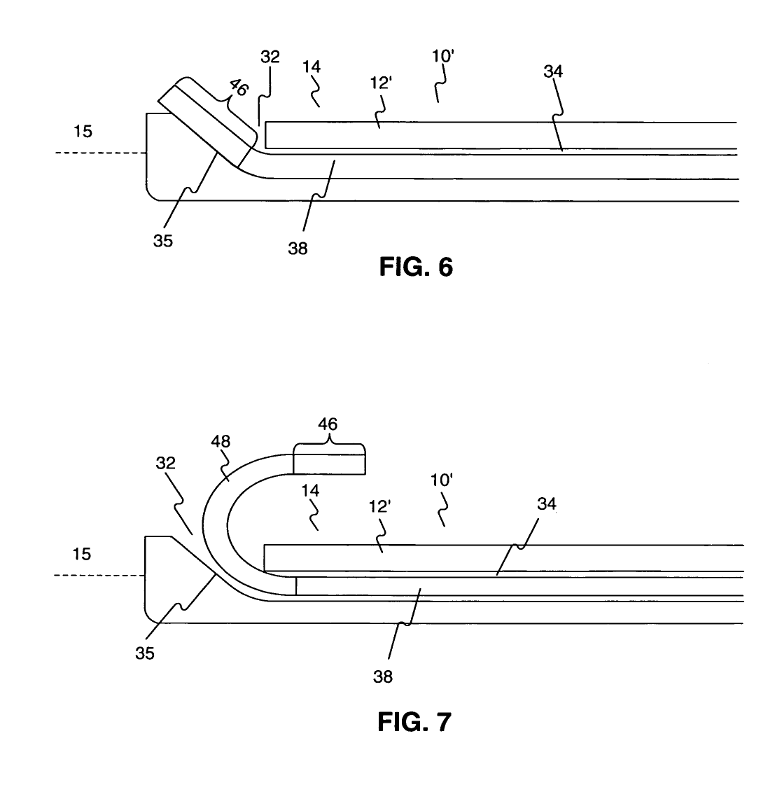

During a medical procedure, the extent to which elevator 38 is deployed beyond the working channel 34 of the underlying endoscope controls the degree of deflection elevator 38 exhibits relative to the longitudinal axis 15 of the endoscope 10′. FIG. 6, for example, depicts a cross-sectional view of a distal end 14 of endoscope 10′ illustrating elevator 38 in a partially extended position. Upon extension beyond working channel 34 and outside aperture 32, the distal-most segment 46 of elevator 38 is no longer restrained by the internal surface of working channel 34. Accordingly, the distal portion 46 is free to revert substantially back to any initial trained shape when no longer housed within working channel 34. In the illustrated embodiment of elevator 38, the distal-most segment comprises a relatively linear shape. Therefore, in the elevator's limited extended position of FIG. 6, the guide path for a treatment instrument corresponds to that of the path along ramp 35 within endoscope 10′ leading out of aperture 32. Alternatively, in an endoscope with a forward facing aperture 32, for example, extension of elevator 38 to the degree illustrated in FIG. 6 would result in no deflection of a guide path along the elevator 38.

FIG. 7 depicts elevator 38 in a further extended position. As seen in FIG. 7, continued deployment of elevator 38 beyond working channel 34 exposes a portion of intermediate segment 48 beyond working channel 34 and aperture 32. The deployed portion of intermediate segment 48 exhibits its unrestrained curved shape, thereby altering the angle at which the exposed portion of elevator 38 deflects relative to the longitudinal axis 15 of the underlying endoscope 10′. The intermediate segment 48 may be formed to curve through an angular orientation of about 180 degrees, for example. Accordingly, the angle of deflection for elevator 38 can be selectively altered relative to the underlying endoscope's longitudinal axis 15 by an adjustable angle between 0 and 180 degrees.

Therefore, depending on the extent of elevator deployment, an operator can selectively alter the angle at which the elevator 38 is configured to guide a treatment instrument therethrough. Upon reaching a desired deflection angle, an operator can lock or otherwise fix the position of the elevator relative to the working channel 34 such that the initial trained shape of distal and intermediate portions 46 and 48 remain unconstrained beyond aperture 32. An operator can then track a treatment instrument within the conduit 39 (see FIG. 5) of the locked elevator 38 in order to position a treatment instrument along the guide path presented by the deployed portion of elevator 38.

In at least one embodiment, the elevator 38 could form a tube along all, or most of, its length. In this arrangement, either all or simply a terminal portion of the tube's interior could include a grooved conduit 39 for receiving and guiding a treatment instrument therein. The tube forming the elevator 38 can then be manipulated and deployed beyond the working channel of an endoscope for positioning during a procedure. In addition, in every embodiment described in this specification, the elevator 38 could be configured for rotation within, and relative to the longitudinal axis of, the working channel of the underlying endoscope. Therefore, rotation of the elevator, after a deployment beyond the working channel within which it is initially housed, provides an additional positioning capability for an underlying treatment instrument.

While the embodiment of FIGS. 3-7 illustrates a particular arrangement of three segments and a particular shape for elevator 38, alternative shapes are intended to be within the scope of this disclosure. The particular unrestrained, trained shape for elevator 38 can be customized to facilitate access to a particular anatomical treatment location. For example, the trained shape of elevator 38 may comprise a curved shape which deflects laterally and proximally back toward the working channel 34 as depicted in FIG. 7. Alternatively, the trained shape of elevator 38 may comprise a curved shape which deflects laterally and distally beyond the working channel 34. In addition, and as another example, distal-most segment 46 may be eliminated, so that the elevator consists of a curved segment 48 and a proximal linear segment 50.

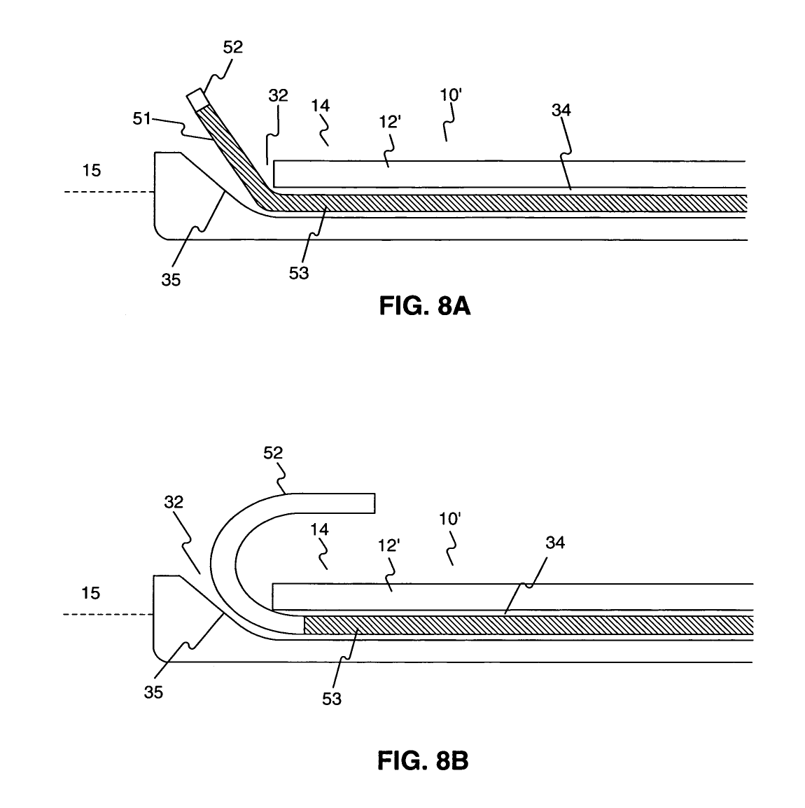

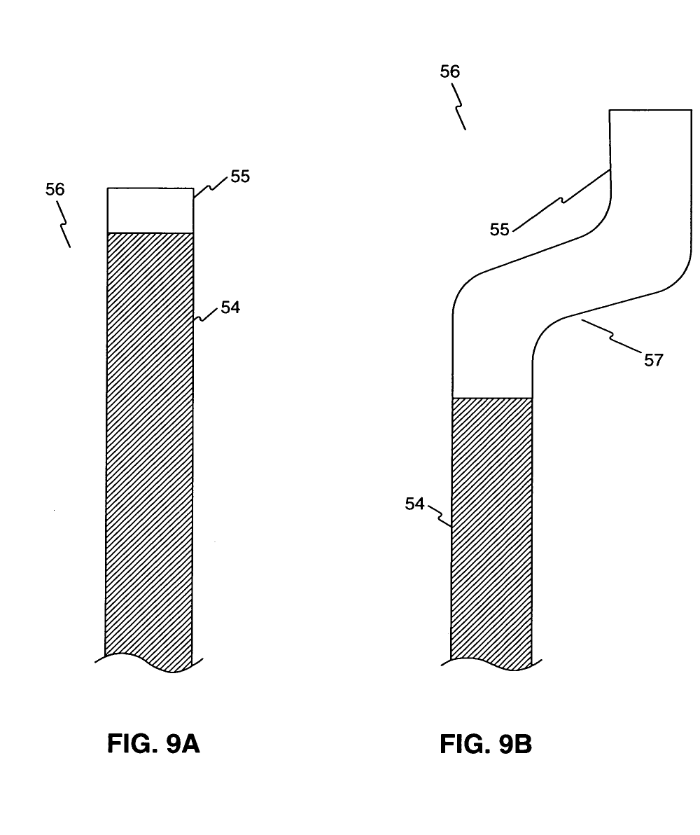

FIGS. 8A-9B depict additional arrangements for an endoscope elevator component. FIG. 8A depicts a cross-sectional view of the endoscope 10′ of FIGS. 3-7, including a different elevator 51 instead of the previously described elevator 38. Just as in the previously described embodiments, the distal portion of endoscope 10′ includes an exterior flexible outer tube 12′, a side facing operation window aperture 32, and a working channel 34 forming a lumen within the endoscope 10′ and extending from the proximal end of the endoscope 10′ and terminating at the operation window aperture 32. The flexible outer tube 12′ extends along a longitudinal axis 15. The working channel 34 is configured to receive a treatment instrument therein. As noted above, while FIGS. 8A-8B illustrate a side facing operation aperture 32, both forward facing and oblique angled embodiments are contemplated.

Channel 34 houses a retractable elongated endoscope elevator 51 therein. Elevator 51 may extend or retract within and relative to channel 34. The elevator 51 is configured to provide a guide path for a treatment instrument in order to alter the path through which the treatment instrument extends outside the endoscope's working channel 34 and aperture 32. As will be described in more detail below, the elongated elevator 51 can be configured so as to form a groove or guide channel along a surface thereof. In the embodiment illustrated in FIGS. 8A-8B, the elevator 51 comprises a tube 52 having a lumen therein forming the guide channel of the elevator 51. The lumen of tube 52 serves to receive and guide a treatment instrument therein and, in particular, directs the placement of a treatment instrument beyond the aperture 32 during a medical procedure. The tube 52 is surrounded by and slidably received within a sleeve 53. The sleeve 53 and tube 52 together comprise the elevator 51. As will be described in more detail below, the shape of the elevator 51 can be altered upon the retraction of sleeve 53 relative to tube 52.

For example, in FIG. 8A, the elevator 51 is depicted as extending slightly beyond the aperture 32. The tube 52, just as the elongated elevator 38 in the previous embodiments, can comprise a shape-memory material formed into a first “trained” shape that is retained during resting conditions (e.g., when the shaped material is in free space or when external forces applied to the shaped material are insufficient to substantially deform the shape). The tube 52, therefore, can be deformed into additional shapes when subjected to a sufficiently strong external force and revert substantially back to the initial, first trained shape when external forces are no longer applied.

In the illustrated embodiment, when the sleeve 53 is positioned to surround the tube 52 (as in FIG. 8A with the exception of a small distal portion), the sleeve 53 imparts a predetermined rigidity to the elevator 51, thereby deforming the tube 52. The deformation of tube 52 results in a second shape for the elevator 51 when it is extended beyond and no longer restrained by the working channel 34. As seen in FIG. 8A, the elevator 51 extends from the aperture 32 at an approximately 45 degree angle. While a 45 degree angle for elevator 51 is depicted, other configurations are contemplated and the invention should not be limited to any one particular arrangement.

FIG. 8B depicts elevator 51 in a further deployed condition. In FIG. 8B, the elevator 51 is depicted after the retraction of sleeve 53 relative to the tube 52. As seen in FIG. 8B, sleeve 53 is retracted proximally relative to the tube 52 such that the entire distal end of sleeve 53 extends into the working channel 34. Upon the retraction of sleeve 53 relative to tube 52, the tube 52 then reverts to the first “trained” shape that is retained during resting conditions. In the configuration illustrated in FIG. 8B, tube 52 reverts to, a curved shape where the tube 52 deflects toward the proximal end of the endoscope 10′. Therefore, upon controlled retraction of sleeve 53 relative to tube 52, an operator can control the change in configuration for the elevator 51. Such control thereby allows an operator to more precisely control the placement of a treatment instrument guided through the underlying elevator 51.

The elevator can be configured to exhibit various alternative shapes. For example, instead of the previous configuration, the sleeve can be used to restrain lateral movement of an underlying elevator tube when extended to surround the tube. FIGS. 9A-9B depict an example of a sleeve 54 that restrains lateral movement of an underlying elevator tube 55. FIGS. 9A-9B illustrate top views of an elevator arrangement including a tube 55 surrounded by, and slidably received within, sleeve 54. The sleeve 54 and tube 55 together comprise an elevator arrangement 56. As seen in the configuration of FIG. 9A, the sleeve 54 completely surrounds the tube 55 (with the exception of a small portion of the distal end of tube 55), thereby restraining the tube 55 from its initial trained shape. As seen in FIG. 9B, the sleeve 54 is retracted relative to the tube 55, thereby releasing the tube 55 from the external restraining force of sleeve 54. The resting trained shape of elevator tube 55 exhibits a lateral bend 57. In other words, the distal end of the tube is configured for movement in one sideways direction upon the removal of an external restraining force. Accordingly, in the illustrated configuration, when the sleeve 54 is retracted relative to the tube 55, the distal end of the elevator tube 55 can be controlled to move in a lateral direction due to the exposure of the bend 57.

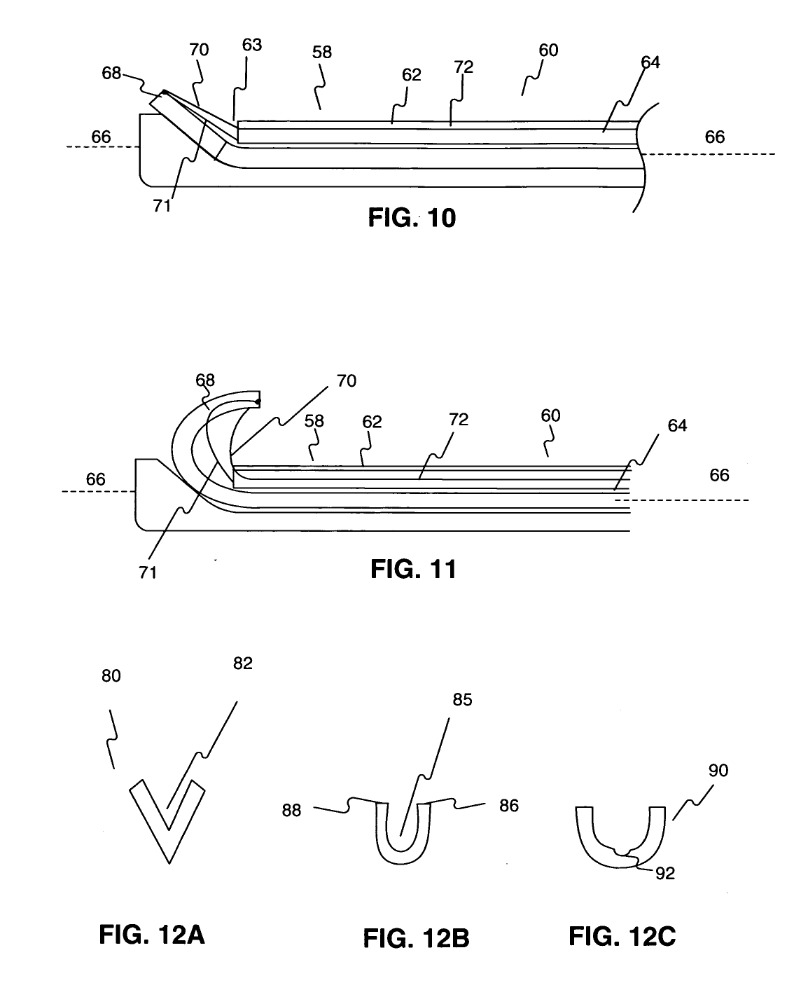

FIG. 10 depicts a cross-sectional view of a distal end 58 of an endoscope 60. The endoscope 60 includes an exterior flexible outer tube 62, a side facing operation window aperture 63, and a working channel 64 forming a lumen within the endoscope 60 and extending from the proximal end of the endoscope 60 and terminating at the operation window aperture 63. The flexible outer tube 62 extends along a longitudinal axis 66. As seen in FIG. 10, the distal end of channel 64 is curved and ramped laterally leading to aperture 63. The working channel 64 is configured to receive an elevator 68 formed of a shape-memory material. Just as in the embodiments of FIGS. 3-7, elevator 68 can be formed into a “trained” shape that is retained during resting conditions. The elevator 68 also can then be deformed into a second shape when subjected to a sufficiently strong external force and revert substantially back to the initial, trained shape when external forces are no longer applied.

The endoscope 60 further includes a pull wire system including, for example, an embodiment of two pull wires 70 and 71 connected to laterally offset positions at a distal end of elevator 68. In addition, the pull wire system can include a single pull wire or greater than two pull wires. In the illustrated embodiment, each pull wire 70 and 71 extends from its point of connection at elevator 68 proximally through separate pull wire lumens 72, only one of which is visible in the side views of FIGS. 10-11. The pull wires 70 and 71 extend through their respective pull wire lumens 72 where they terminate upon connection with a pull wire actuation mechanism, such as, for example, at an endoscope handle at a proximal end of the device.

In one embodiment, the pull wire lumens 72 housing pull wires 70 and 71 are spaced a predetermined lateral distance across the width of and within the flexible outer tube 62. As a result of this lateral spacing, actuation of the first pull wire 70 deflects the distal portion of elevator 68 in one sideways direction, while the actuation of the second pull wire 71 deflects the distal portion of elevator 68 in an opposite sideways direction. For purposes of this disclosure, lateral deflection means the deflection of a distal portion of elevator 68 relative to the longitudinal axis 66 of the tube 62. Lateral deflection, therefore is deflection within the plane of the page of FIGS. 10 and 11, for example. The sideways direction, however, refers to the direction of the width of the flexible tube 62. Deflection in the sideways direction therefore is deflection into and out from the plane of the page of FIGS. 10 and 11, for example. Accordingly, controlled deployment of elevator 68 combined with selective actuation of each pull wire 70 and 71 allows more precise control for laterally deflecting the elevator 68, while also directing movement of the distal end of the elevator 68 in a sideways direction.

The capability of elevator 68 to move laterally through the use of pull wires may be restricted by the distance the pull wires lumens 72 can be spaced laterally relative to the center of outer tube 62. Therefore, the amount of torque acting on elevator 68 can be increased by increasing the moment arm of the system (i.e. the distance pull wire lumens are spaced from the center of tube 62). Alternatively, the resistance to torsion by the elevator 68 can be adjusted by making a section of the elevator more flexible than the majority of the elevator. The elevator can be made more flexible by choice of material (e.g., a material of a lower durometer hardness) or by providing relief cuts into the elevator structure.

FIG. 10 depicts elevator 68 extended outside the working channel 64 of endoscope 10′. FIG. 11 further depicts controlled deflection of the extended elevator 68 through actuation of pull wire 70 in the proximal direction with no concurrent actuation of the additional pull wire 71. Proximal displacement of pull wire 70 draws the distal end of elevator 68 closer to the distal opening of pull wire lumen 72, which, as noted above, is spaced laterally in the sideways direction within the flexible tube 62. Therefore, actuation of pull wire 70 without concurrent actuation of pull wire 71 may (1) control the angle at which elevator 68 extends outside the working channel 64 (i.e. lateral deflection in an amount greater than that of the trained shape retained by elevator 68 during resting conditions) and (2) effectuates partial sideways displacement of the elevator 68 relative to the outer flexible tube 62. An operator may therefore control the placement of a treatment instrument by extending the elevator 68 to a desired limit and then selectively actuating either of pull wires 70 and 71 while fixing the longitudinal position of the proximal portion of elevator 68 within the working channel 64. Therefore, the distal orientation of the elevator 68 can be selectively manipulated without effecting displacement of any remaining portion of the elevator 68 housed within channel 64.

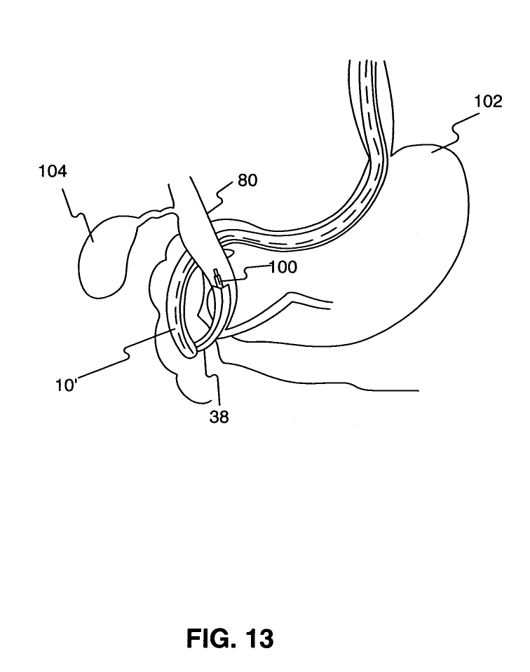

As noted above, the elevators of this disclosure are configured to provide a guide path for a treatment instrument in order to alter the path through which the treatment instrument extends outside an endoscope's working channel. An elongated elevator according to this invention can be configured so as to form a groove or guide channel along a surface thereof, which receives and guides a treatment instrument therein. FIGS. 12A-12C illustrate various alternative shapes for the cross-section of an elevator according to embodiments of the invention. FIG. 12A, for example, depicts a “V” shaped cross-section configuration 80. The internal surface of the v-shaped channel 82 serves to receive and guide a treatment instrument therein. FIG. 12B depicts a “U” shaped cross-section configuration. The u-shaped configuration houses and guides a treatment instrument within the channel 85 formed between the two extending edges 86 and 88 of the configuration. In addition, FIG. 12C illustrates a concave shaped cross-section configuration 90. The internal concave surface of the elevator configuration 90 further includes a recess channel 92 configured to receive an external surface of a treatment instrument therein. The recess channel 92 can be precisely customized to correspond to the external shape of a particular treatment instrument or can be generically sized to permit a broad range of treatment instrument sizes therein.

FIG. 13 illustrates the positioning of an endoscope 10′ or 60 and a treatment device 100 within a patient's body portion. In particular, FIG. 13 depicts the extension of a treatment instrument 100 within a particular bile duct during an ERCP procedure. As seen in FIG. 13, the endoscope 10′, for example, is inserted and extended through a patient's stomach 102 such that the distal end and aperture 32 (not shown) of endoscope 10′ are positioned is close relation to a particular bile duct 80 leading to, for example, gall bladder 104. As seen in FIG. 13, elevator 38 is extended beyond the internal working channel of endoscope 10′. By controlling the extent to which elevator 38 is deployed beyond the working channel of endoscope 10′, an operator controls the angle at which elevator 38 deflects relative to the longitudinal axis of endoscope 10′. Upon reaching a desired deployment configuration for elevator 38, the treatment instrument 100 can then be inserted through the working channel of the endoscope 10′ and guided along the v-shaped internal guide conduit 39 of the elevator 38 depicted in FIG. 13. Accordingly, a treatment instrument 100 can then be more precisely directed to a particular treatment location.

Precise manipulation of elevator 38 allows for more precise positioning and location of instrument 100 such as, for example, during placement of instrument 100 within a particular bile duct of interest. More precise manipulation of a treatment device 100 can result in shortened treatment procedures by reducing the amount of time necessary to effectuate proper position of the treatment device 100. In addition, controlled deflection of the angle at which treatment device 100 exits the underlying endoscope 10′ can reduce internal tissue trauma caused during endoscopic procedures requiring repeated repositioning and manipulation of the entire endoscope during location of the endoscope. For example, the positioning mechanisms described in FIGS. 3-12C facilitate the location of treatment instrument 100 within a particular bile duct such that the duration of, and occurrence of tissue trauma during, a treatment procedure can be reduced.

In addition to the positioning mechanisms disclosed above, the system of this application may further include other additional positioning mechanisms, such as those for achieving controlled deflection of the elongated flexible tube of the endoscope.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A medical device, comprising:
an elongated flexible tube including a distal end, a proximal end, and a channel extending from the proximal end to an aperture proximate to the distal end; and
an elongated elevator positioned within the channel and movable relative to the channel, the elevator having a first shape when the elevator is within the channel of the tube and a second shape when the elevator extends out of the aperture beyond the channel, wherein the elevator is configured to receive and direct a treatment instrument for placement at a treatment location.

2. The medical device of claim 1, wherein the elevator is formed of a shape memory material.

3. The medical device of claim 1, wherein the elevator is configured to retain the second shape without the application of a force to the elevator.

4. The medical device of claim 1, wherein the elevator is configured to retain the first shape when a force is applied to the elevator.

5. The medical device of claim 4, wherein the force is applied by the tube.

6. The medical device of claim 1, wherein the first shape of the elevator corresponds to an internal shape of the channel in the tube and the second shape of the elevator comprises a curved shape.

7. The medical device of claim 6, wherein the aperture is a side facing aperture opening laterally along the flexible tube and the curved shape comprises a curve with a distal end which deflects proximally relative to the aperture of the flexible tube.

8. The medical device of claim 6, wherein the aperture is a side facing aperture opening laterally along the flexible tube and the curved shape comprises a curve with a distal end which deflects distally relative to the aperture of the flexible tube.

9. The medical device of claim 1, wherein the second shape of the elevator comprises a distal linear portion and an intermediate curved portion.

10. The medical device of claim 9, wherein the intermediate curved portion comprises a curve extending at least 90 degrees and exhibiting a substantially constant radius of curvature.

11. The medical device of claim 1, wherein the elevator has a V-shaped cross-section.

12. The medical device of claim 1, wherein the elevator has a U-shaped cross-section.

13. The medical device of claim 1, wherein the elevator has a recess channel configured to receive an external surface of a treatment instrument therein.

14. The medical device of claim 1, wherein the elevator is configured for sideways deflection when extended beyond the channel of the tube through actuation of a pull wire connected to a distal portion of the elevator and extending proximally within the medical device.

15. The medical device of claim 14, wherein two pull wires are connected to a distal portion of the elevator and extend proximally within laterally offset lumens within the tube.

16. The medical device of claim 1, wherein the medical device is an endoscope that includes visualization and illumination components therein.

17. The medical device of claim 1, wherein the medical device is an endoscope that includes an additional positioning mechanism for achieving controlled deflection of the elongated flexible tube.

18. The medical device of claim 1, further comprising a handle at the proximal end of the flexible tube, the handle including a positioning mechanism connected to the elevator and for extending and retracting the elevator within the channel of the flexible tube.

19. The medical device of claim 18, wherein the positioning mechanism comprises a slide block connected to a proximal portion of the elevator, the slide block extending within a slide channel formed along an exterior surface of the handle.

20. The medical device of claim 19, wherein the slide channel includes boundaries limiting the distance the slide block can extend and retract the elevator.

21. The medical device of claim 18, wherein the positioning mechanism includes a locking mechanism for fixing the position of the elevator relative to the channel of the flexible tube.

22. The medical device of claim 1, wherein the elevator exhibits a third shape when the elevator extends out of the aperture beyond the channel and a restraining force is removed from the elevator.

23. The medical device of claim 22, wherein the first shape of the elevator corresponds to an internal shape of the channel in the tube, the second shape of the elevator comprises a second curved shape, and the third shape of the elevator comprises a third curved shape different than the second curved shape.

24. The medical device of claim 1, wherein the elevator comprises a hollow tube configured for receiving a treatment instrument therein.

25. The medical device of claim 23, wherein the elevator comprises a hollow tube slidably received within a sleeve.

26. The medical device of claim 25, wherein the sleeve imparts rigidity to the hollow tube when surrounding the hollow tube, thereby restraining the hollow tube to exhibit the second curved shape when the elevator extends out of the aperture beyond the channel.

27. The medical device of claim 26, wherein upon extension of the elevator beyond the aperture and upon retraction of the sleeve relative to the hollow tube, the elevator exhibits the third curved shape.

28. The medical device of claim 22, wherein the first shape of the elevator corresponds to an internal shape of the channel in the tube, the second shape of the elevator comprises a curved shape, and the third shape of the elevator comprises a shape having a lateral bend.

29. A method of positioning a treatment instrument in a body comprising:
providing a medical device comprising:
an elongated flexible tube including a distal end, a proximal end, and a channel extending from the proximal end to an aperture proximate to the distal end; and
an elongated elevator positioned within the channel and movable relative to the channel, the elevator having a first shape when the elevator is within the channel of the tube and a second shape when the elevator extends out of the aperture beyond the channel, wherein the elevator is configured to receive and direct a treatment instrument for placement at a treatment location;
inserting the medical device into an anatomical lumen of the body;
extending the elevator beyond the channel of the tube such that the elevator achieves the second shape; and
inserting a treatment instrument along the elevator such that the treatment instrument is positioned at the treatment location.

30. The method of claim 29, further comprising retracting the treatment instrument into the medical device, retracting the elevator into the channel of the tube, repositioning the medical device within the anatomical lumen, and redeploying the elevator and treatment instrument.

31. The method of claim 29, wherein the medical device includes a handle having a positioning mechanism for extending, retracting, and locking the position of the elevator within the channel of the flexible tube; and further comprising locking the position of the elevator within the channel of the flexible tube.

32. The method of claim 29, wherein the treatment instrument is positioned within a bile duct during an ERCP procedure.

33. The method of claim 29, wherein the medical device is an endoscope that includes visualization and illumination components therein.

34. The method of claim 29, wherein the medical device is an endoscope that includes an additional positioning mechanism for achieving controlled deflection of the elongated flexible tube.

35. The method of claim 29, wherein the elevator is formed of a shape memory material.

36. The method of claim 29, wherein the elevator is configured to retain the second shape without the application of a force to the elevator.

37. The method of claim 29, wherein the elevator is configured to retain the first shape when a force is applied to the elevator.

38. The method of claim 37, wherein the force is applied by the tube.

39. The method of claim 29, wherein the first shape of the elevator corresponds to an internal shape of the channel in the tube and the second shape of the elevator comprises a curved shape.

40. The method of claim 29, wherein the elevator has a V-shaped cross-section.

41. The method of claim 29, wherein the elevator has a U-shaped cross-section.

42. The method of claim 29, wherein the elevator deflects sideways when extended beyond the channel of the tube through actuation of a pull wire connected to a distal portion of the elevator and extending proximally within the medical device.

43. The method of claim 42, wherein two pull wires are connected to a distal portion of the elevator and extend proximally within laterally offset lumens within the tube.

44. The method of claim 29, wherein the elevator exhibits a third shape when the elevator extends from the aperture beyond the channel and a restraining force is removed from the elevator.

45. The method of claim 44, wherein the elevator comprises a hollow tube slidably received within a sleeve.

46. The method of claim 45, wherein the sleeve imparts a rigidity to the hollow tube when surrounding the hollow tube, thereby restraining the hollow tube to exhibit the second shape when the elevator extends from the aperture beyond the channel.

47. The method of claim 46, further comprising extending the elevator beyond the elongated flexible tube and retracting the sleeve relative to the hollow tube such that the elevator exhibits the third shape.

48. The method of claim 44, wherein the first shape of the elevator corresponds to an internal shape of the channel in the tube, the second shape of the elevator comprises a curved shape, and the third shape of the elevator comprises a shape having a lateral bend.
